(19) United States
(12) Patent Application Publication
(10) Pub. No.: US 2025/0213195 A1
(43) Pub. Date: Jul. 3, 2025

(54) BRAIN WAVE DETECTION SYSTEM, BRAIN WAVE DETECTION METHOD AND NON-TRANSITORY STORAGE MEDIUM STORING NOISE REDUCTION PROCESSING PROGRAM

(71) Applicant: TOYOTA JIDOSHA KABUSHIKI KAISHA, Toyota-shi (JP)

(72) Inventors: Keiji HAYASHI, Toyota-shi (JP); Hitoshi YAMADA, Toyota-shi (JP); Yuhei YAMAGUCHI, Toyota-shi (JP)

(73) Assignee: TOYOTA JIDOSHA KABUSHIKI KAISHA, Toyota-shi (JP)

(21) Appl. No.: 18/987,601

(22) Filed: Dec. 19, 2024

(30) Foreign Application Priority Data
Dec. 27, 2023 (JP) 2023-221199

(51) Int. Cl. A61B 5/00; A61B 5/029; A61B 5/369

(52) U.S. Cl. CPC A61B 5/7203; A61B 5/029; A61B 5/369; A61B 5/7225

(57) ABSTRACT

A brain wave detection system according to the present embodiment includes: a brain wave measurer that measures brain waves; a pulse wave measurer that measures pulse waves; and a noise reduction processing device that performs a process of reducing noise in the measured brain waves by comparing a time-series signal of the measured brain waves, as a measurement signal, with a time-series signal of the measured pulse waves, as a reference signal, the time-series signal of the pulse waves being measured in a measurement time slot corresponding to a measurement time slot of the time-series signal of the brain waves.

### CROSS-REFERENCE TO RELATED APPLICATION

This application claims priority to Japanese Patent Application No. 2023-221199 filed on Dec. 27, 2023, incorporated herein by reference in its entirety.

### BACKGROUND

### 1. Technical Field

The present disclosure relates to a brain wave detection system, a brain wave detection method and a non-transitory storage medium storing a noise reduction processing program.

### 2. Description of Related Art

Japanese Unexamined Patent Application Publication No. 2017-189471 (JP 2017-189471 A) discloses a brain wave detection device that corrects measured brain waves by using sensor values from a sensor for sensing myoelectric potential caused by body motion, such as clenching of teeth or blinking.

### SUMMARY

Although myoelectric noise may arise from all head and neck muscles, it is difficult for the brain wave detection device of JP 2017-189471 A to place sensors on all the muscles. Accordingly, it is difficult to reduce noise caused by a muscle on which a sensor for sensing myoelectric potential is not placed.

The present disclosure has been made to solve such a problem and provides a brain wave detection system, a brain wave detection method and a non-transitory storage medium storing a noise reduction processing program that make it possible to reduce noise included in measured brain waves.

A brain wave detection system according to the present embodiment includes: a brain wave measurer that measures brain waves; a pulse wave measurer that measures pulse waves; and a noise reduction processing device that performs a process of reducing noise in the measured brain waves by comparing a time-series signal of the measured brain waves, as a measurement signal, with a time-series signal of the measured pulse waves, as a reference signal, the time-series signal of the pulse waves being measured in a measurement time slot corresponding to a measurement time slot of the time-series signal of the brain waves. With such a configuration, noise included in measured brain waves can be reduced.

In the brain wave detection system, the noise reduction processing device may perform the process of reducing the noise in the brain waves by filtering the measured brain waves and the measured pulse waves with at least one frequency band, and comparing the time-series signal of the filtered brain waves, as the measurement signal, with the time-series signal of the filtered pulse waves, as the reference signal, the time-series signal of the pulse waves being measured in the measurement time slot corresponding to the measurement time slot of the time-series signal of the brain waves. With such a configuration, noise in brain waves can be reduced in each frequency band.

In the brain wave detection system, the pulse wave measurer may measure the pulse waves at at least any one of a carotid arterial bifurcation and a subclavian arterial bifurcation. With such a configuration, pulse waves closely relating to brain waves can be measured.

In the brain wave detection system, the noise reduction processing device may perform the process of reducing the noise in the measured brain waves by performing a lock-in amplification process. With such a configuration, noise can be reduced through the lock-in amplification process.

A brain wave detection method according to the present embodiment includes: measuring brain waves; measuring pulse waves; and performing a process of reducing noise in the measured brain waves by comparing a time-series signal of the measured brain waves, as a measurement signal, with a time-series signal of the measured pulse waves, as a reference signal, the time-series signal of the pulse waves being measured in a measurement time slot corresponding to a measurement time slot of the time-series signal of the brain waves. With such a configuration, noise included in measured brain waves can be reduced.

A non-transitory storage medium storing a noise reduction processing program according to the present embodiment causes a computer to execute a procedure for performing a process of reducing noise in measured brain waves by comparing a time-series signal of the measured brain waves, as a measurement signal, with a time-series signal of pulse waves, as a reference signal, the time-series signal of the pulse waves being measured in a measurement time slot corresponding to a measurement time slot of the time-series signal of the brain waves. With such a configuration, noise included in measured brain waves can be reduced.

According to the embodiment, a brain wave detection system, a brain wave detection method and a non-transitory storage medium storing a noise reduction processing program can be provided that make it possible to reduce noise included in measured brain waves.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 illustrates a configuration of a brain wave detection system according to a first embodiment;

FIG. 2 is a block diagram illustrating a noise reduction processing device according to the first embodiment;

FIG. 3 is a flowchart illustrating a brain wave detection method according to the first embodiment;

FIG. 4 is a graph illustrating a time-series signal of pulse waves according to the first embodiment, wherein a horizontal axis represents time and a vertical axis represents intensity;

FIG. 5 is a flowchart illustrating a noise reduction processing method performed by the noise reduction processing device according to the first embodiment;

FIG. 6 is a graph illustrating signal waveforms after pulse waves are filtered in individual frequency bands according to the first embodiment, wherein a horizontal axis represents time and a vertical axis represents intensity;

FIG. 7 illustrates the individual frequency bands used when brain waves and pulse waves are filtered according to the first embodiment;

FIG. 8 is a graph illustrating signal waveforms after pulse waves are Fourier-transformed according to the first embodiment, wherein a horizontal axis represents frequency and a vertical axis represents spectral power;

FIG. 9 is a graph illustrating brain waves and pulse waves according to the first embodiment, wherein a horizontal axis represents time and a vertical axis represents intensity;

FIG. 10 is a graph illustrating brain waves and pulse waves according to the first embodiment, wherein a horizontal axis represents time and a vertical axis represents intensity;

FIG. 11 is a graph illustrating brain waves and pulse waves according to the first embodiment, wherein a horizontal axis represents time and a vertical axis represents intensity;

FIG. 12 is a graph illustrating brain waves and pulse waves according to the first embodiment, wherein a horizontal axis represents time and a vertical axis represents intensity;

FIG. 13 is a graph illustrating brain waves and pulse waves according to the first embodiment, wherein a horizontal axis represents time and a vertical axis represents intensity;

FIG. 14 is a graph illustrating brain waves and pulse waves according to the first embodiment, wherein a horizontal axis represents time and a vertical axis represents intensity;

FIG. 15 is a graph illustrating brain waves and pulse waves according to the first embodiment, wherein a horizontal axis represents time and a vertical axis represents intensity;

FIG. 16 is a graph illustrating brain waves and pulse waves according to the first embodiment, wherein a horizontal axis represents time and a vertical axis represents intensity;

FIG. 17 is a graph illustrating brain waves and pulse waves according to the first embodiment, wherein a horizontal axis represents time and a vertical axis represents intensity;

FIG. 18 is a graph illustrating brain waves and pulse waves according to the first embodiment, wherein a horizontal axis represents time and a vertical axis represents intensity;

FIG. 19 is a graph illustrating brain waves and pulse waves according to the first embodiment, wherein a horizontal axis represents time and a vertical axis represents intensity; and

FIG. 20 is a graph illustrating brain waves and pulse waves according to the first embodiment, wherein a horizontal axis represents time and a vertical axis represents intensity.

### DETAILED DESCRIPTION OF EMBODIMENTS

Hereinafter, embodiments are described through the present disclosure. However, the scope of claims is not limited to the embodiment described below. Not all the components described in the embodiment are necessarily needed as means for solving the problem. For a clear description, omission and simplification are made as appropriate in the description below and the drawings. Throughout the drawings, the same elements are denoted by the same signs, and duplicate explanation is omitted as necessary.

### First Embodiment

A brain wave detection system according to a first embodiment is described. The brain wave detection system of the present embodiment performs a process of reducing noise in measured brain waves by comparing a time-series signal of measured brain waves, as a measurement signal, with a time-series signal of pulse waves measured in a measurement time slot corresponding to that of the time-series signal of brain waves, as a reference signal. In other words, a signal-to-noise ratio, as the amplitude of the brain wave signal, is improved by using the pulse wave signal. Hereinafter, first, a configuration of the brain wave detection system is described. Thereafter, a brain wave detection method using the brain wave detection system is described.

### Configuration of Brain Wave Detection System

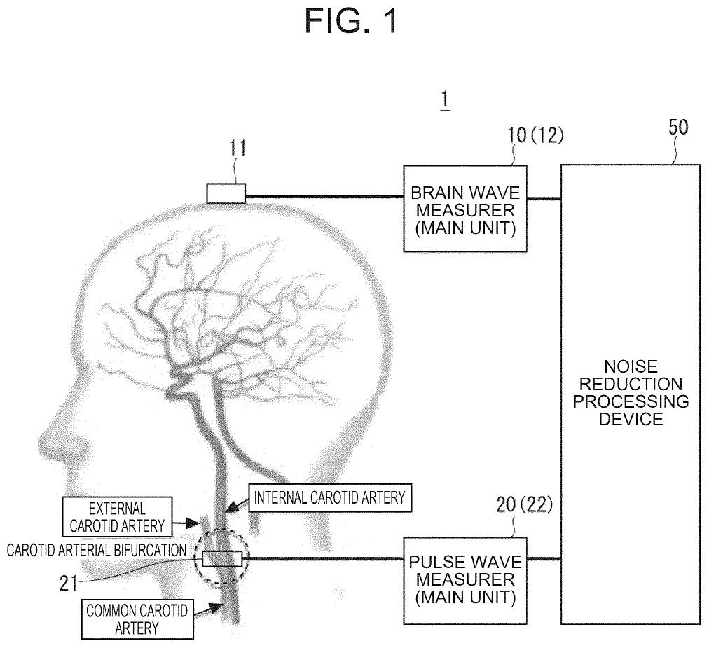

FIG. 1 illustrates the configuration of the brain wave detection system according to the first embodiment. As shown in FIG. 1, the brain wave detection system 1 includes a brain wave measurer 10, a pulse wave measurer 20, and a noise reduction processing device 50.

The brain wave measurer 10 measures brain waves of a living body. Specifically, the brain wave measurer 10 measures a time-series signal of brain waves. The living body is, for example, a human being examined. The brain wave measurer 10 includes a sensor 11 and a main unit 12. For example, the sensor 11 is attached onto the scalp of a human head and senses information on human brain waves from the outside of the living body. The information on human brain waves is, for example, electric voltage. Note that apart from the electric voltage, the sensor 11 may sense electric current, a magnetic field, or the like as information on human brain waves. The sensor 11 is placed on the living body in a non-invasive manner.

The sensor 11 outputs the sensed information on brain waves to the main unit 12 of the brain wave measurer 10. The main unit 12 of the brain wave measurer 10 measures changes over time in the voltage or the like output from the sensor 11. The sensor 11 is connected to the main unit 12 through a wired or wireless communication link. The main unit 12 is connected to the noise reduction processing device 50 through a wired or wireless communication link. The main unit 12 outputs the measured brain waves to the noise reduction processing device 50.

The pulse wave measurer 20 measures pulse waves of the living body. Specifically, the pulse wave measurer 20 measures a time-series signal of pulse waves. The pulse waves are waveform information on the living body. The waveform information is made of pulse intervals, blood output volumes, and physical characteristics of blood vessels. The pulse wave measurer 20 includes a sensor 21 and a main unit 22. For example, the sensor 21 is attached to a skin in a human neck region or on a clavicle and measures information on human pulse waves from the outside of the living body. Specifically, the sensor 21 may be placed in at least any one of vicinities of the bifurcations of the right and left carotid arteries and vicinities of the bifurcations of the right and left subclavian arteries. The information on human pulse waves is, for example, pulse pressure. Note that apart from the pulse pressure, the sensor 21 may sense a blood flow amount or the like as information on human pulse waves. For example, a photoplethysmography-based pulse wave measurer may be used for sensing. The sensor 21 is placed on the living body in a non-invasive manner.

The brain wave detection system 1 of the present embodiment is a technology that uses a state in which brain waves and pulse waves correlate. Accordingly, it is preferable that places to acquire pulse waves be the bifurcations of the right and left carotid arteries and the bifurcations of the right and left subclavian arteries. A large vessel carrying blood that flows into the brain branches at a carotid arterial bifurcation and a subclavian arterial bifurcation. Beyond such branches are the frontal lobe, the parietal site, and the like. A site of the brain desired to be observed is, for example, the frontal lobe and the parietal site. Accordingly, it is preferable that pulse waves be measured at the bifurcations of the right and left carotid arteries and the bifurcations of the right and left subclavian arteries. Thus, pulse waves relating to blood flows in the internal carotid arteries and the vertebral arteries can be measured.

The sensor 21 outputs the sensed information on pulse waves to the main unit 22 of the pulse wave measurer 20. The main unit 22 of the pulse wave measurer 20 measures changes over time in the pulse pressure or the like output from the sensor 21. The sensor 21 is connected to the main unit 22 through a wired or wireless communication link. The main unit 22 is connected to the noise reduction processing device 50 through a wired or wireless communication link. The main unit 22 outputs the measured pulse waves to the noise reduction processing device 50.

The noise reduction processing device 50 uses the measured time-series signal of brain waves for a measurement signal. Moreover, the noise reduction processing device 50 uses a time-series signal of pulse waves measured in a measurement time slot corresponding to that of the time-series signal of brain waves, for a reference signal. The noise reduction processing device 50 performs a process of reducing noise in the measured brain waves by comparing the measurement signal with the reference signal. Specifically, the noise reduction processing device 50 separates each of the brain wave signal and the pulse wave signal into individual frequency bands by using bandpass filters. Thereafter, the noise reduction processing device 50 makes such an adjustment as to make the measurement time slot of the pulse waves correspond to the measurement time slot of the brain waves by temporally delaying the pulse wave signal. Moreover, the noise reduction processing device 50 adjusts the amplitude components of the brain waves and the amplitude components of the pulse waves. The noise reduction processing device 50 performs the process of reducing noise in the measured brain waves by performing a lock-in amplification process using the measurement signal and the reference signal.

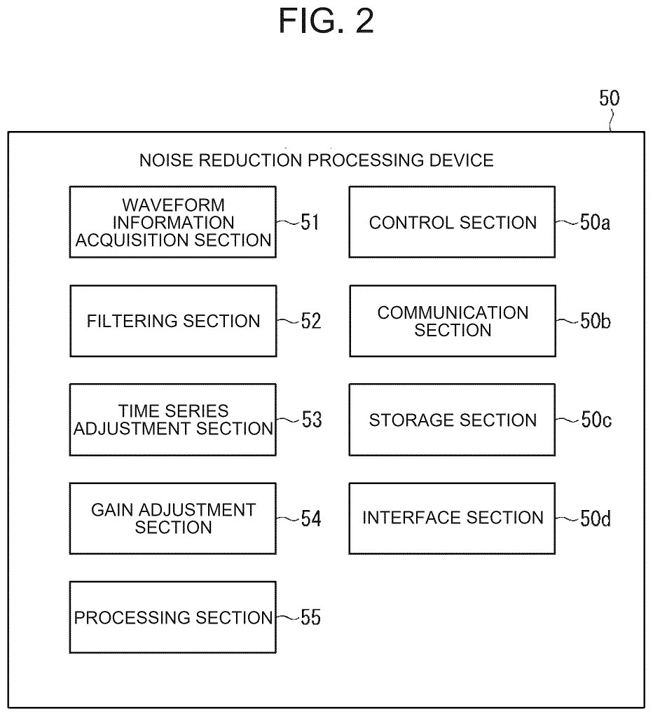

FIG. 2 is a block diagram illustrating the noise reduction processing device 50 according to the first embodiment. As shown in FIG. 2, the noise reduction processing device 50 includes a control section 50a, a communication section 50b, a storage section 50c, an interface section 50d, a waveform information acquisition section 51, a filtering section 52, a time series adjustment section 53, a gain adjustment section 54, and a processing section 55. The control section 50a, the communication section 50b, the storage section 50c, the interface section 50d, the waveform information acquisition section 51, the filtering section 52, the time series adjustment section 53, the gain adjustment section 54, and the processing section 55 have functionality as control means, communication means, storage means, interface means, waveform information acquisition means, filtering means, time series adjustment means, gain adjustment means, and processing means, respectively.

The noise reduction processing device 50 is an information processing device including a computer. The control section 50a includes a processor, such as a central processing unit (CPU), a micro processing unit (MPU), an electronic control unit (ECU), a field-programmable gate array (FPGA), or an application specific integrated circuit (ASIC). The control section 50a has functionality as an arithmetic-logic unit that performs control processing, calculation processing, and the like. The control section 50a controls operation of each of the constituent elements, such as the communication section 50b, the storage section 50c, the interface section 50d, the waveform information acquisition section 51, the filtering section 52, the time series adjustment section 53, the gain adjustment section 54, and the processing section 55.

For example, each constituent element of the noise reduction processing device 50 can be implemented by executing a program through control by the control section 50a. The program is stored in a storage medium. More specifically, each constituent element can be implemented by the control section 50a executing a program stored in the storage section 50c. A necessary program may be stored in an arbitrary non-volatile recording medium beforehand, and each constituent element may be implemented by installing the program as necessary. Not limited to being implemented by software by using a program, each constituent element may be implemented by a combination, or the like, of any of hardware, firmware, and software.

The communication section 50b receives the time-series signal of brain waves measured by the brain wave measurer 10 and the time-series signal of pulse waves measured by the pulse wave measurer 20 from the brain wave measurer 10 and the pulse wave measurer 20, respectively.

The storage section 50c may include a storage device, such as a memory or a hard disk. The storage device is, for example, a read only memory (ROM), a random access memory (RAM), or the like. The storage section 50c has a function for storing a control program, a calculation program, and the like that are executed by the control section 50a. The storage section 50c has a function for temporarily storing processed data and the like. The storage section 50c may store the time-series signals of brain waves and pulse waves received by the communication section 50b.

The interface section 50d is, for example, a user interface. The interface section 50d includes an input device, such as a key board, a touch panel, or a mouse, and an output device, such as a display or a speaker. The interface section 50d receives a data input operation performed by a user (an operator or the like) and outputs information to the user.

The waveform information acquisition section 51 acquires the time-series signals of brain waves and pulse waves that the communication section 50b acquires from the brain wave measurer 10 and the pulse wave measurer 20, respectively. The filtering section 52 filters the measured brain waves and the measured pulse waves with at least one frequency band. Accordingly, the noise reduction processing device 50 may perform the noise reduction process by using a time-series signal of the filtered brain waves and a time-series signal of the filtered pulse waves.

The time series adjustment section 53 adjusts time series between the time-series signal of brain waves and the time-series signal of pulse waves. Specifically, the time series adjustment section 53 acquires the time-series signal of pulse waves that is acquired in a measurement time slot corresponding to that of the time-series signal of brain waves. Note that to make the measurement time slots of brain waves and pulse waves correspond to each other, for example, a delay of pulse waves corresponding to brain waves may be measured beforehand, or a signal serving as a mark may be selected beforehand.

The gain adjustment section 54 adjusts gains (amplitudes) between the time-series signal of brain waves and the time-series signal of pulse waves. The processing section 55 compares the measured time-series signal of brain waves, as a measurement signal, with the time-series signal of pulse waves acquired in a measurement time slot corresponding to that of the time-series signal of brain waves, as a reference signal. Thus, the processing section 55 performs the process of reducing noise in the measured brain waves. For example, the processing section 55 performs the lock-in amplification process by using the measured brain waves for a measurement signal and the measured pulse waves for a reference signal.

### Brain Wave Detection Method

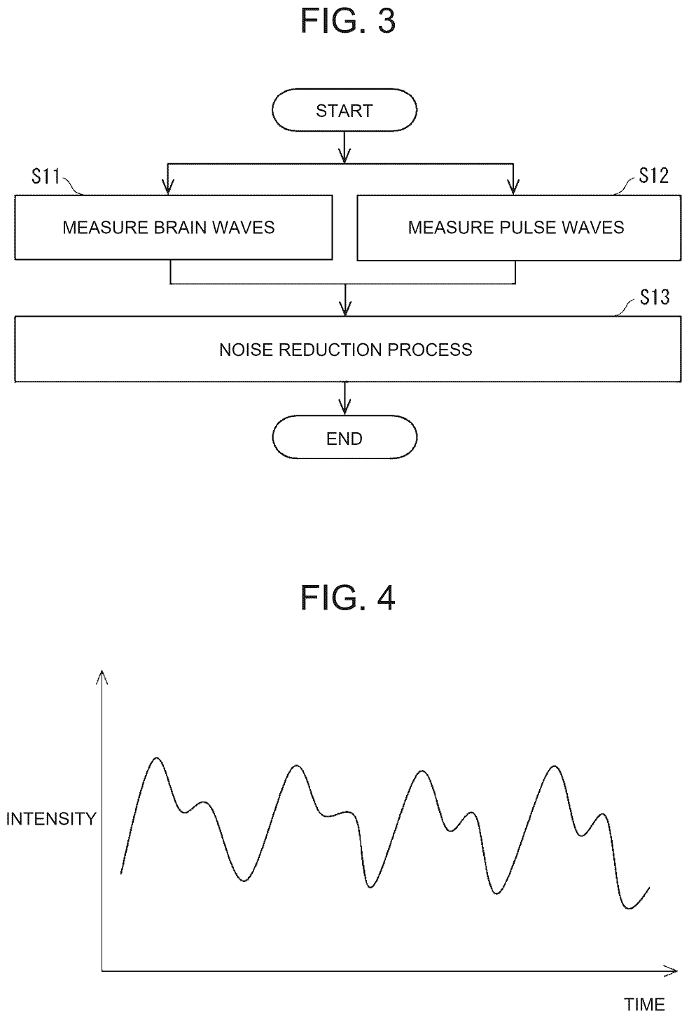

Next, the brain wave detection method is described. FIG. 3 is a flowchart illustrating the brain wave detection method according to the first embodiment. As shown in FIG. 3, the brain wave detection method includes a brain wave measurement step of measuring brain waves (step S11), a pulse wave measurement step of measuring pulse waves (step S12), and a noise reduction processing step of performing the process of reducing noise in the brain waves (step S13).

As shown at step S11 in FIG. 3, brain waves are measured. For example, the brain wave measurer 10 measures brain waves of a living body. As shown at step S12, pulse waves are measured. For example, the pulse wave measurer 20 measures pulse waves of the living body. It is preferable that step S11 and step S12 be performed at the same time. However, order in which step S11 and step S12 are performed is not limited because time series are adjusted between the brain waves and the pulse waves in a time series adjustment step (step S23), which will be descried later. In other words, step S11 may be performed before step S12, or may be performed after step S12.

FIG. 4 is a graph illustrating a time-series signal of pulse waves according to the first embodiment, wherein a horizontal axis represents time and a vertical axis represents intensity. As shown in FIG. 4, the pulse wave measurer 20 measures a time-series signal of pulse waves. It is preferable that the pulse wave measurer 20 measure pulse waves at, for example, at least any one of the bifurcations of the right and left carotid arteries and the bifurcations of the right and left subclavian arteries of a human being examined. Pulse waves are observed when a photoplethysmography-based pulse wave measurer is applied to a carotid arterial bifurcation and a subclavian arterial bifurcation. The pulse waves there are thought to be almost proportional to the amount of blood that flows into the brain. Accordingly, the pulse waves measured from a carotid arterial bifurcation and a subclavian arterial bifurcation closely relate to brain waves. Hence, noise in the brain waves can be reduced through the noise reduction processing using the pulse waves for a reference signal.

A time-series signal of pulse waves is acquired via the sensor 21 placed at at least any one of the bifurcations of the carotid arteries and the bifurcations of the subclavian arteries. For example, pulse waves of the human being examined in a stationary state are measured for several minutes to several hours. Sampling frequency is, for example, 500 Hz. For the sampling frequency, any frequency in a range of 10 to 1000 Hz can be used. A pulse wave signal thus acquired has repetitive waveforms as in FIG. 4. The pulse wave measurer 20 outputs the acquired pulse waves to the noise reduction processing device 50.

Next, as shown at step S13, the process of reducing noise in the measured brain waves is performed by comparing the measured time-series signal of brain waves, as a measurement signal, with the time-series signal of pulse waves acquired in a measurement time slot corresponding to that of the measured time-series signal of brain waves, as a reference signal.

### Noise Reduction Processing Method

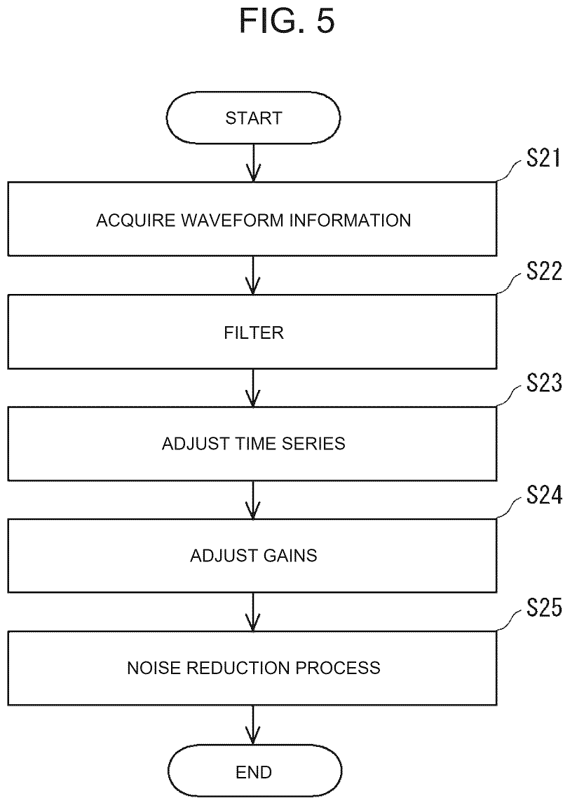

Next, a noise reduction processing method in step S13 described above is described. FIG. 5 is a flowchart illustrating the noise reduction processing method performed by the noise reduction processing device 50 according to the first embodiment. As shown in FIG. 5, the noise reduction processing method includes a waveform information acquisition step of acquiring brain waves and pulse waves (step S21), a filtering step of filtering the acquired waveform information with at least one frequency band (step S22), a time series adjustment step of making time series correspond to each other by delaying one of the filtered waveform information in the frequency band (step S23), a gain adjustment step of adjusting a gain of one of the waveform information (step S24), and a noise reduction processing step of performing the process of reducing noise in the brain waves (step S25). Hereinafter, each step is described.

### Waveform Information Acquisition Step

The waveform information acquisition section 51 of the noise reduction processing device 50 acquires the brain waves and the pulse waves that the communication section 50b receives from the brain wave measurer 10 and the pulse wave measurer 20, respectively. For example, the waveform information acquisition section 51 acquires the time-series signals of brain waves and pulse waves from the communication section 50b.

### Filtering Step

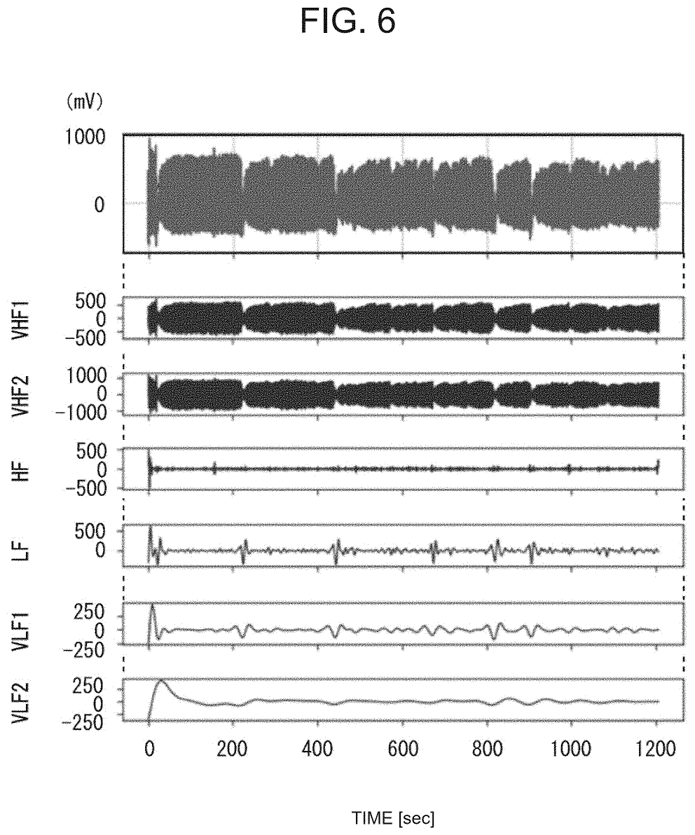

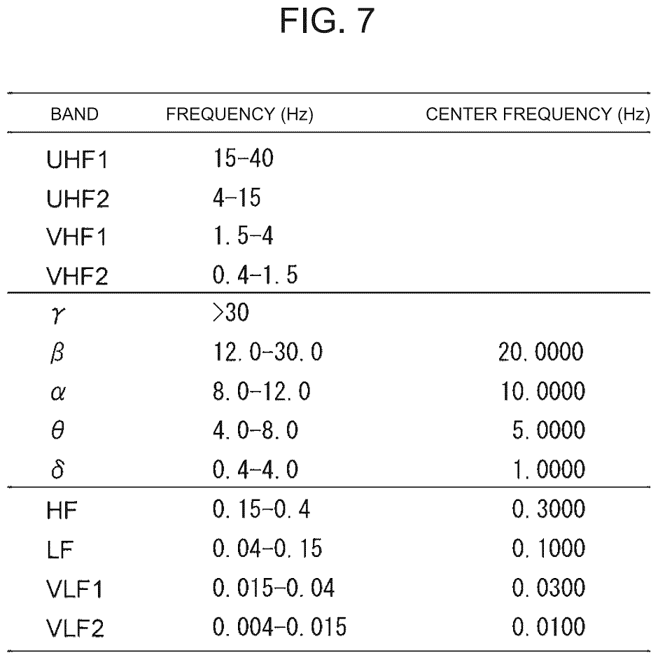

FIG. 6 is a graph illustrating signal waveforms after pulse waves are filtered with individual frequency bands according to the first embodiment, wherein a horizontal axis represents time and a vertical axis represents intensity. In FIG. 6, HF, LF, VLF1, VLF2, VHF1 and VHF2 are shown as the individual frequency bands. Moreover, pulse waves before filtered are also shown in FIG. 6. FIG. 7 illustrates the individual frequency bands used when waveform information relating to a living body is filtered according to the first embodiment. The center frequencies of some frequency bands are also shown in FIG. 7.

As shown in FIG. 7, the filtering section 52 of the noise reduction processing device 50 filters the acquired brain waves and pulse waves with at least one frequency band. Specifically, the filtering section 52 filters the brain waves and the pulse waves acquired by the waveform information acquisition section 51, with each frequency band.

As shown in FIGS. 6 and 7, for frequency bands used for filtering, for example, VLF2 (0.004 to 0.015 Hz), VLF1 (0.015 to 0.04 Hz), LF (0.04 to 0.15 Hz), HF (0.15 to 0.4 Hz), VHF2 (0.4 to 1.5 Hz), VHF1 (1.5 to 4 Hz), UHF2 (4 to 15 Hz), UHF1 (15 to 40 Hz), and the like are selected. Here, in the brain, there are neural activity networks and activity centers that are activated in various frequency bands. For example, HF is of the respiratory center and is a respiratory variable band with heartbeat intervals. LF is of the blood pressure center and is a blood pressure variable band. The VHF2, VHF1, UHF2 and UHF1 bands include, of the brain waves, a δ-wave (0.4 to 4.0 Hz) band for a sleep network, a θ-wave (4.0 to 8.0 Hz) band for a meditation network, an α-wave (8.0 to 12.0 Hz) band for a relaxation network, a β-wave (12.0 to 30.0 Hz) band, and a γ-wave (over 30 Hz) band. Brain activities can be minutely estimated by observing a brain blood flow and brain waves in each of the specific frequency bands.

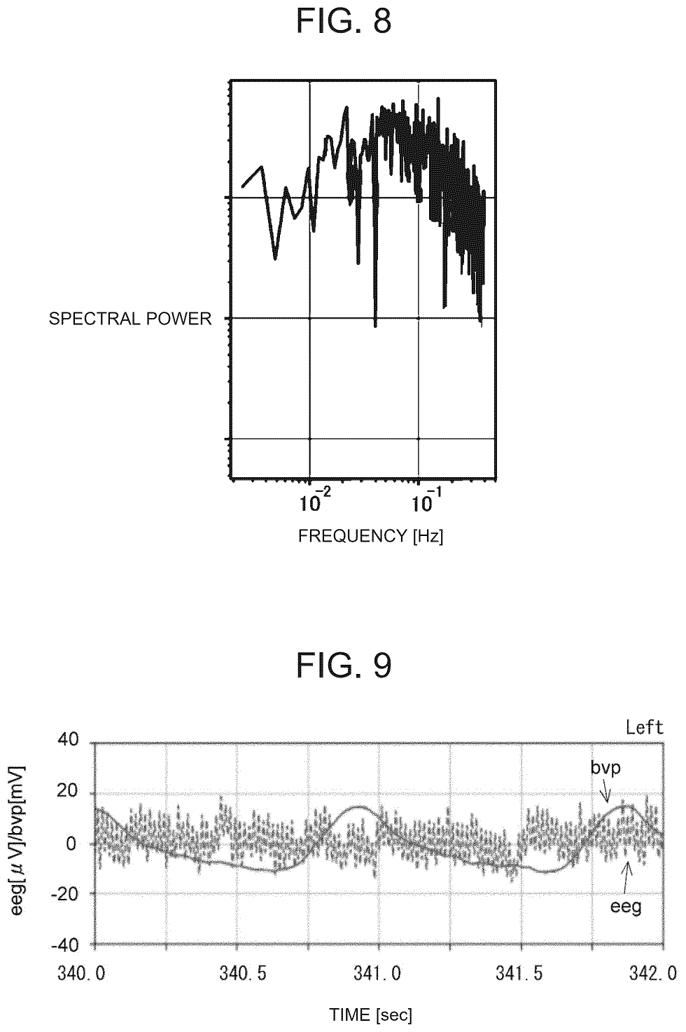

FIG. 8 is a graph illustrating signal waveforms after pulse waves are Fourier-transformed according to the first embodiment, wherein a horizontal axis represents frequency and a vertical axis represents spectral power. As shown in FIG. 8, after Fourier-transformed, the pulse waves in FIG. 4 or the like may be filtered with the individual frequency bands. Thereafter, inverse Fourier transform may be performed.

### Time Series Adjustment Step

The time series adjustment section 53 of the noise reduction processing device 50 adjusts measurement time slots between the filtered time-series signal of brain waves in each frequency band and the filtered time-series signal of pulse waves in each frequency band. For example, one of the brain waves and the pulse waves is delayed with respect to the other. Thus, the time series adjustment section 53 acquires the time-series signal of pulse waves acquired in a measurement time slot corresponding to that of the time-series signal of brain waves.

### Gain Adjustment Step

The gain adjustment section 54 of the noise reduction processing device 50 adjusts gains (amplitudes) between the filtered time-series signal of brain waves in each frequency band and the filtered time-series signal of pulse waves in each frequency band. For example, the gain of one of the brain waves and the pulse waves is increased with respect to the other. Thus, the gain adjustment section 54 adjusts the gains of the brain waves and the pulse waves.

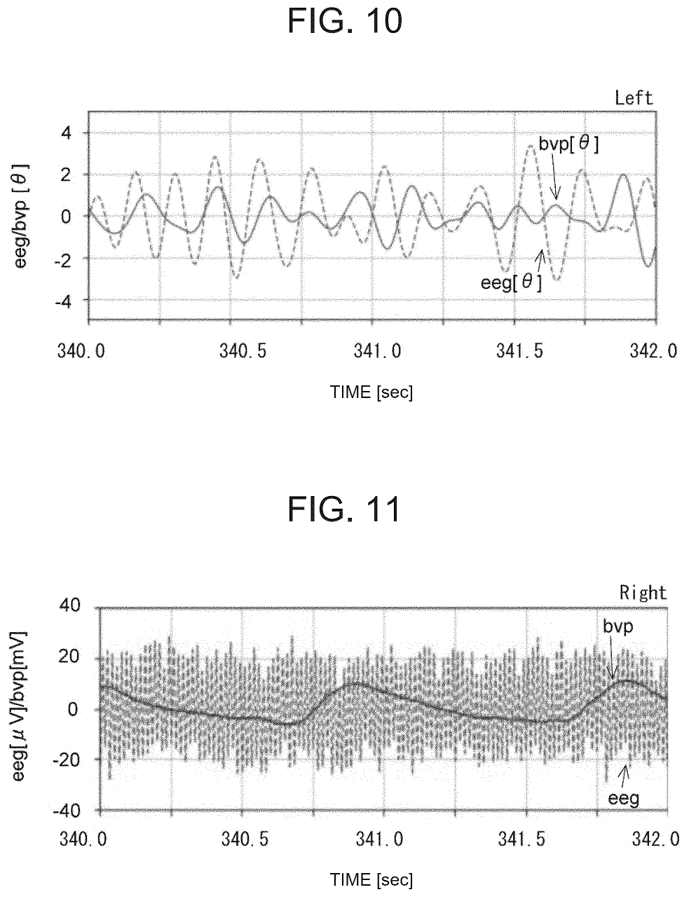

FIGS. 9 and 10 are graphs illustrating brain waves and pulse waves according to the first embodiment, wherein a horizontal axis represents time and a vertical axis represents intensity. The brain waves are indicated by eeg, and the pulse waves are indicated by bvp. In FIGS. 9 and 10, brain waves from the left brain and pulse waves at the left-side carotid arterial bifurcation are shown. FIG. 10 shows the brain waves and pulse waves that are filtered in the θ-wave band as a frequency band and adjusted in time series and gain.

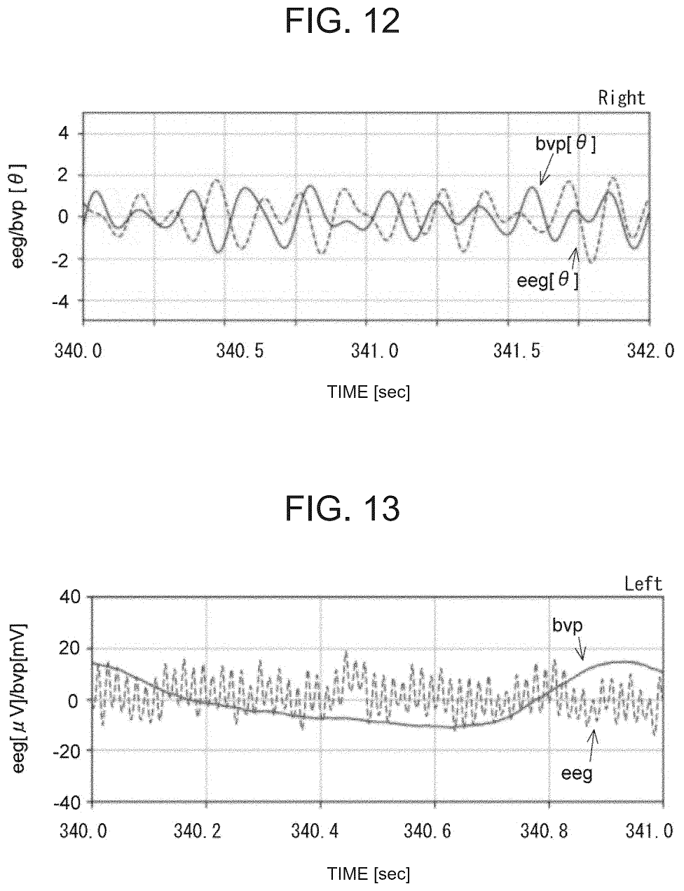

FIGS. 11 and 12 are graphs illustrating brain waves and pulse waves according to the first embodiment, wherein a horizontal axis represents time and a vertical axis represents intensity. In FIGS. 11 and 12, brain waves from the right brain and pulse waves at the right-side carotid arterial bifurcation are shown. FIG. 12 shows the brain waves and pulse waves that are filtered in the θ-wave band as a frequency band and adjusted in time series and gain.

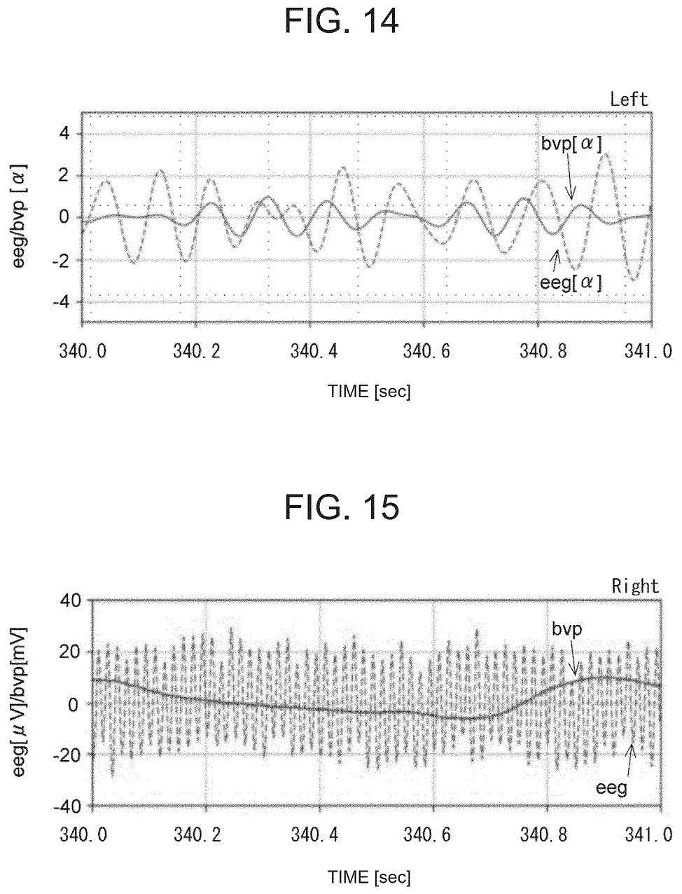

FIGS. 13 and 14 are graphs illustrating brain waves and pulse waves according to the first embodiment, wherein a horizontal axis represents time and a vertical axis represents intensity. In FIGS. 13 and 14, brain waves from the left brain and pulse waves at the left-side carotid arterial bifurcation are shown. FIG. 14 shows the brain waves and pulse waves that are filtered in the α-wave band as a frequency band and adjusted in time series and gain.

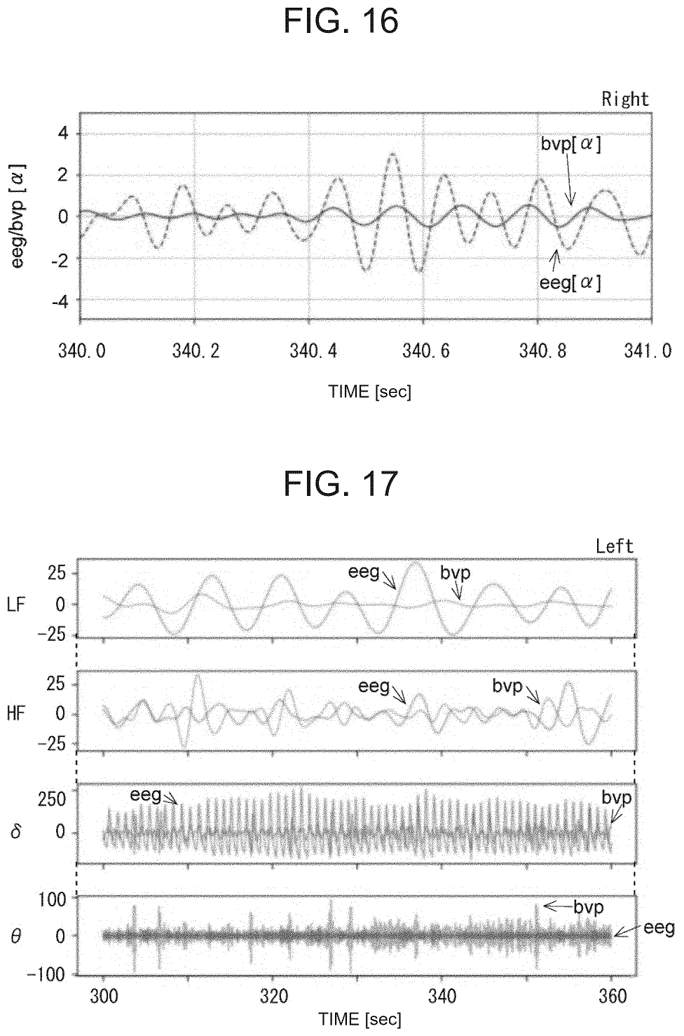

FIGS. 15 and 16 are graphs illustrating brain waves and pulse waves according to the first embodiment, wherein a horizontal axis represents time and a vertical axis represents intensity. In FIGS. 15 and 16, brain waves from the right brain and pulse waves at the right-side carotid arterial bifurcation are shown. FIG. 16 shows the brain waves and pulse waves that are filtered in the α-wave band as a frequency band and adjusted in time series and gain.

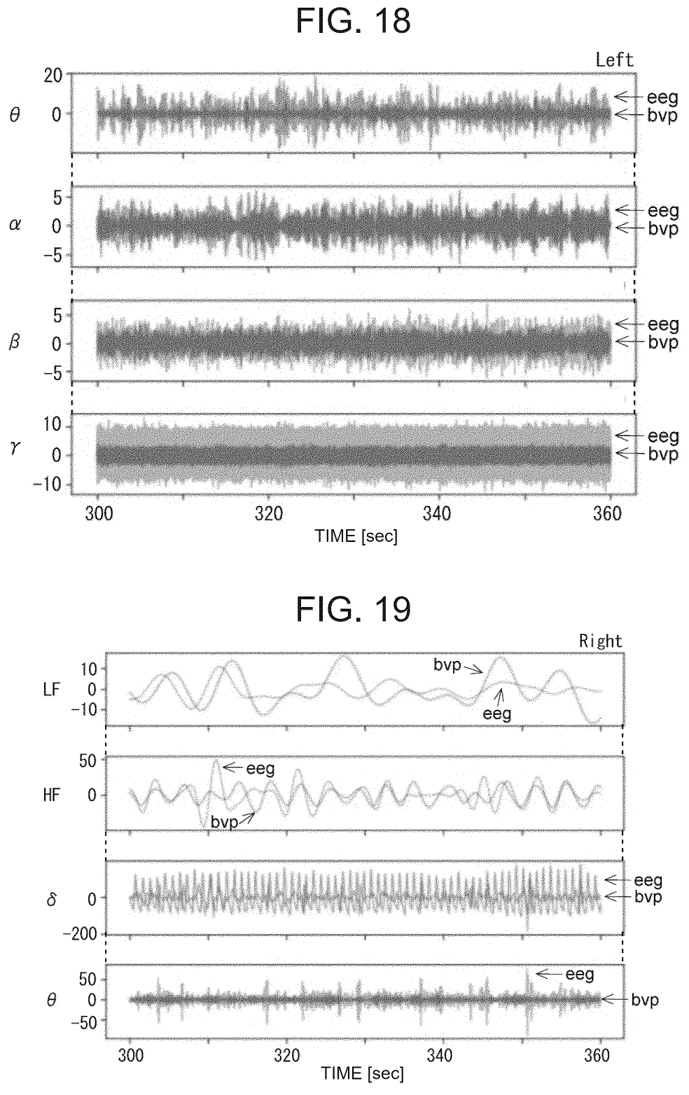

FIGS. 17 and 18 are graphs illustrating brain waves and pulse waves according to the first embodiment, wherein a horizontal axis represents time and a vertical axis represents intensity. In FIGS. 17 and 18, brain waves from the left brain and pulse waves at the left-side carotid arterial bifurcation are shown. FIGS. 17 and 18 show the brain waves and pulse waves that are filtered in the LF, HF, δ-wave, θ-wave, α-wave, β-wave and γ-wave bands as frequency bands and adjusted in time series and gain. For the 0 waves, brain waves and pulse waves with different gains are shown.

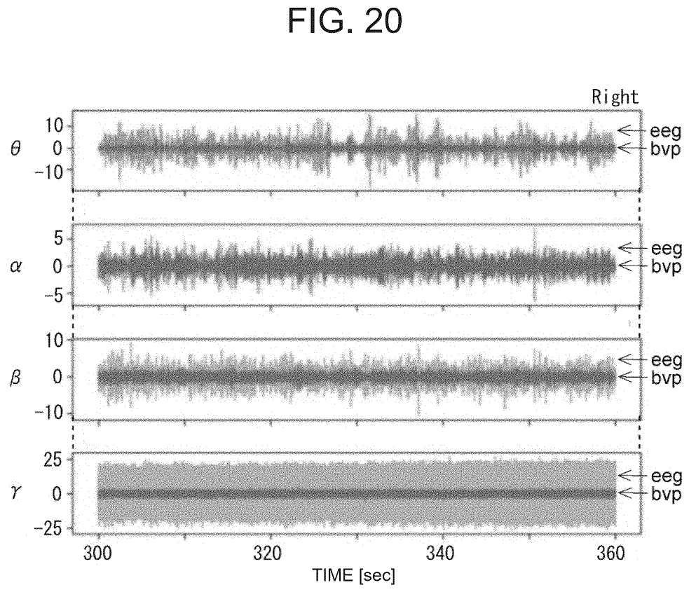

FIGS. 19 and 20 are graphs illustrating brain waves and pulse waves according to the first embodiment, wherein a horizontal axis represents time and a vertical axis represents intensity. In FIGS. 19 and 20, brain waves from the right brain and pulse waves at the right-side carotid arterial bifurcation are shown. FIG. 19 and FIG. 20 show the brain waves and pulse waves that are filtered in the LF, HF, δ-wave, θ-wave, α-wave, β-wave and γ-wave bands as frequency bands and adjusted in time series and gain. For the 0 waves, brain waves and pulse waves with different gains are shown.

### Noise Reduction Process

The processing section 55 of the noise reduction processing device 50 reduces noise in the brain waves by comparing the measured brain waves, as a measurement signal, with the measured pulse waves, as a reference signal. Specifically, the processing section 55 reduces noise in the measured brain waves by performing the lock-in amplification process. When performing the process of reducing noise in the measured brain waves, the processing section 55 uses a time-series signal of the filtered brain waves for a measurement signal, uses a time-series signal of the filtered pulse waves that are acquired in a measurement time slot corresponding to that of the time-series signal of brain waves for a reference signal, and compares the measurement signal with the reference signal. Thus, the process of reducing noise in the brain waves is performed. At the time, the brain waves and the pulse waves that are adjusted in gain may be used. The processing section 55 can reduce noise in the brain waves shown in FIGS. 9 to 20 by performing the noise reduction process. Thus, the brain wave detection system 1 can detect brain waves in which noise is reduced more than the waveforms of the brain waves shown in FIGS. 9 to 20.

Next, advantageous effects of the present embodiment are described. The brain wave detection system 1 of the present embodiment performs a process of reducing noise in brain waves by comparing a time-series signal of brain waves, as a measurement signal, with a time-series signal of pulse waves, as a reference signal. Accordingly, noise included in the measured brain waves can be reduced.

For brain waves measured in conventional brain wave measurement, it is difficult to improve the signal-to-noise ratio due to effects of environmental noise and living body noise, such as myoelectricity associated with body motion. The brain wave detection system 1 of the present embodiment uses not only brain waves but also pulse waves. In the first place, measured brain waves are based on changes over time in electric potential arising from the activities of nerve cells in the brain. Energy is required for the activities of nerve cells in the brain. Oxygen and glucose that produce such energy are supplied into activated areas of the brain through blood. Accordingly, as the activities of nerve cells in the brain intensify, the amount of blood into an activated area increases. Accordingly, brain waves and the amount of blood (pulse waves) must synchronize with each other. Accordingly, brain waves and pulse waves can be compared as a measurement signal and a reference signal, respectively, and noise included in the brain waves can be reduced.

The noise reduction processing device 50 filters the measured brain waves and pulse waves with individual frequency bands. Accordingly, noise in the brain waves can be reduced in each frequency band. For example, the noise reduction processing device 50 performs the lock-in amplification process by using the measurement signal and the reference signal that are obtained by filtering brain waves and pulse waves by using respective bandpass filters of δ, θ, α, β, γ bands. Thus, the noise reduction processing device 50 can reduce environmental noise and living body noise, such as myoelectricity, and can detect brain waves with a very high signal-to-noise ratio.

Note that the present disclosure is not limited to the embodiment described above, and changes can be made as appropriate to an extent that does not depart from the gist of the present disclosure. For example, a noise reduction processing method and a noise reduction processing program that causes a computer to perform the noise reduction processing method as described below are also included in the technical idea of the present embodiment.

### Supplement 1

A brain wave detection method including: measuring brain waves; measuring pulse waves; and performing a process of reducing noise in the measured brain waves by comparing a time-series signal of the measured brain waves, as a measurement signal, with a time-series signal of the measured pulse waves, as a reference signal, the time-series signal of the pulse waves being measured in a measurement time slot corresponding to a measurement time slot of the time-series signal of the brain waves.

### Supplement 2

The brain wave detection method according to supplement 1, wherein in performing the process of reducing the noise in the measured brain waves, the process of reducing the noise in the brain waves is performed by filtering the measured brain waves and the measured pulse waves with at least one frequency band, and comparing the time-series signal of the filtered brain waves, as the measurement signal, with the time-series signal of the filtered pulse waves, as the reference signal, the time-series signal of the pulse waves being measured in the measurement time slot corresponding to the measurement time slot of the time-series signal of the brain waves.

### Supplement 3

The brain wave detection method according to supplement 1 or 2, wherein in measuring the pulse waves, the pulse waves are measured at at least any one of a carotid arterial bifurcation and a subclavian arterial bifurcation.

### Supplement 4

The brain wave detection method according to any one of supplements 1 to 3, wherein in performing the process of reducing the noise in the measured brain waves, the process of reducing the noise in the measured brain waves is performed by performing a lock-in amplification process.

### Supplement 5

A non-transitory storage medium storing a noise reduction processing program causing a computer to execute a procedure for performing a process of reducing noise in measured brain waves by comparing a time-series signal of the measured brain waves, as a measurement signal, with a time-series signal of pulse waves, as a reference signal, the time-series signal of the pulse waves being measured in a measurement time slot corresponding to a measurement time slot of the time-series signal of the brain waves.

### Supplement 6

The noise reduction processing program according to supplement 5, wherein in the procedure for performing the process of reducing the noise in the measured brain waves, the computer is caused to execute the process of reducing the noise in the brain waves by filtering the measured brain waves and the measured pulse waves with at least one frequency band, and comparing the time-series signal of the filtered brain waves, as the measurement signal, with the time-series signal of the filtered pulse waves, as the reference signal, the time-series signal of the pulse waves being measured in the measurement time slot corresponding to the measurement time slot of the time-series signal of the brain waves.

### Supplement 7

The noise reduction processing program according to supplement 5 or 6, wherein the pulse waves are the pulse waves measured at at least any one of a carotid arterial bifurcation and a subclavian arterial bifurcation.

### Supplement 8

The noise reduction processing program according to any one of supplements 5 to 7, wherein in the procedure for performing the process of reducing the noise in the measured brain waves, the computer is caused to execute the process of reducing the noise in the measured brain waves by performing a lock-in amplification process.

What is claimed is:

1. A brain wave detection system comprising:
a brain wave measurer that measures brain waves;
a pulse wave measurer that measures pulse waves; and
a noise reduction processing device that performs a process of reducing noise in the measured brain waves by comparing a time-series signal of the measured brain waves, as a measurement signal, with a time-series signal of the measured pulse waves, as a reference signal, the time-series signal of the pulse waves being measured in a measurement time slot corresponding to a measurement time slot of the time-series signal of the brain waves.

2. The brain wave detection system according to claim 1, wherein the noise reduction processing device performs the process of reducing the noise in the brain waves by filtering the measured brain waves and the measured pulse waves with at least one frequency band, and comparing the time-series signal of the filtered brain waves, as the measurement signal, with the time-series signal of the filtered pulse waves, as the reference signal, the time-series signal of the pulse waves being measured in the measurement time slot corresponding to the measurement time slot of the time-series signal of the brain waves.

3. The brain wave detection system according to claim 1, wherein the pulse wave measurer measures the pulse waves at at least any one of a carotid arterial bifurcation and a subclavian arterial bifurcation.

4. The brain wave detection system according to claim 1, wherein the noise reduction processing device performs the process of reducing the noise in the measured brain waves by performing a lock-in amplification process.

5. A brain wave detection method comprising:
measuring brain waves;
measuring pulse waves; and
performing a process of reducing noise in the measured brain waves by comparing a time-series signal of the measured brain waves, as a measurement signal, with a time-series signal of the measured pulse waves, as a reference signal, the time-series signal of the pulse waves being measured in a measurement time slot corresponding to a measurement time slot of the time-series signal of the brain waves.

6. A non-transitory storage medium storing a noise reduction processing program causing a computer to execute a procedure for performing a process of reducing noise in measured brain waves by comparing a time-series signal of the measured brain waves, as a measurement signal, with a time-series signal of pulse waves, as a reference signal, the time-series signal of the pulse waves being measured in a measurement time slot corresponding to a measurement time slot of the time-series signal of the brain waves.
